(19) United States
(12) Patent Application Publication
(10) Pub. No.: US 2005/0267410 A1
(43) Pub. Date: Dec. 1, 2005

(54) Needlestick prevention device

(76) Inventor: Marc Koska, East Sussex (GB)

Correspondence Address: MICHAEL BEST & FRIEDRICH LLC, 401 NORTH MICHIGAN AVENUE, SUITE 1900, CHICAGO, IL, 60611-4212, US

(21) Appl. No.: 10/517,885

(22) PCT Filed: Jun. 13, 2003
(86) PCT No.: PCT/GB03/02546

§ 371 Date: Apr. 25, 2005

(30) Foreign Application Priority Data
Jun. 13, 2002 (GB) 0213555.6

(51) Int. Cl. A61M 37/00

(52) U.S. Cl. 604/110; 604/198

(57) ABSTRACT

A needlestick prevention device for an injection device (1) having a hollow needle (2) comprises a sheath having a first member (9) for attachment to the injection device (1) and a second member (10) slidable longitudinally relative to the first member (9) to expose or to cover the needle (2), and spring means (11) biasing the second member (10) to cover the needle (2). The first and second members (9, 10) have interengaging guide means (13) and locking means (14), including a first guide part (23) operative to allow free longitudinal sliding movement of the second member (10) relative to the first member (9), and a second guide part (24) operative on movement by manual relative rotation of the first and second members (9, 10) and following release of a force urging the second member (10) to expose the needle (2). The spring means (11) urges the second member (10) to cover the needle (2) and to operate the locking means (14) to retain the second member (10) covering the needle (2). This allows free movement of the second member (10) in the first guide part (23), allowing for filling of the syringe (1), but then automatic sheathing and locking when the user simply twists the second member (10) relative to the first (9).

This invention relates to a needlestick prevention device for use with injection devices, such as syringes.

A needlestick injury generally occurs in a medical environment, and particularly before or after use of a syringe or other injection device, when the user accidentally sticks the needle into himself or herself, or indeed another person. It is of course important to prevent such injuries, since they can spread infections and diseases, as well as being painful and possibly incapacitating.

There are several known ways of trying to prevent needlestick. For example, some syringes are made with needles which are retracted automatically after use by means of a spring in the syringe. However, this requires a complex construction, and is expensive to manufacture. Further, it does not address the possibility of injury occurring before the injection is given.

Another known way is to replace the tubular sheath which is supplied with the syringe, protecting the needle. It is not now recommended practice to replace the sheath after the syringe has been used, because of the difficulty of placing the end of the needle accurately in the sheath. It is thought that trying to re-sheath the needle has actually been the cause of a significant number of needlestick injuries. Further, the sheath can easily be removed again, so that injury is possible.

Yet another known needlestick prevention device is an automatic needle sheath mounted on the syringe barrel, the sheath being able to slide out to cover the needle. EP-A-0 268 445 shows a construction with a stationary sheath part, and a movable sheath part spring-biassed to an extended position in which it covers the needle. The movable sheath part retracts to expose the needle for use, and when the retracting pressure is released the spring moves it automatically into its extended position, where it is locked. The movable sheath part has a projection received in a track in the stationary part to determine its movement and to lock it. However in one embodiment the track is such that the movable sheath part can be unlocked by twisting it relative to the stationary part, and in another its movement is dictated by the track, so that the user has no choice about its use.

According to the present invention, a needlestick prevention device for an injection device having a hollow needle comprises a sheath having a first member for attachment to the injection device and a second member slidable longitudinally relative to the first member to expose or to cover the needle, and spring means biassing the second member to cover the needle, the first and second members having interengaging guide means and locking means, including a first guide part operative to allow free longitudinal sliding movement of the second member relative to the first member, and a second guide part operative on movement by manual relative rotation of the first and second members and following release of a force urging the second member to expose the needle, in which the spring means urges the second member to cover the needle and to operate the locking means to retain the second member covering the needle.

Thus, the invention allows free movement of the second member in the first guide part, allowing for filling of the syringe, expulsion of air and finding of an injection site (or a site for taking a blood sample), but then automatic sheathing and locking in the second guide part, provided by the spring means. The user simply has to twist the second member relative to the first to cause the automatic sheathing and locking to operate. This provides the necessary element of choice for the user as to when to sheath and lock the needle, while being safe to use, as the simple twisting movement carries no danger of needlestick. Indeed, the twisting can be carried out when the needle has been inserted into the patient. The device of the invention is therefore simple to use and to manufacture, as it has only three components.

The guide means preferably comprises at least one groove means on one of the first and second members, and a corresponding projection on the other of the members which slides in the groove means. Preferably two groove means and projections are provided, arranged in diametral opposition. Conveniently the second member slides inside the first member. The or each groove means is then provided on the radially exterior surface of the second member and the or each projection on the radially interior surface of the first member. This arrangement simplifies manufacture. It would of course be possible for the groove means to be on the first member and the projection on the second member.

In the or each guide means the first guide part preferably comprises a first groove extending longitudinally of the second member. The second guide part comprises a second groove extending longitudinally of the second member. The second groove is preferably parallel to the first, and spaced from it such that a relative rotation of 30° of the members will move the projection from the first groove into the second groove. The locking means conveniently comprises a permanent locking recess formed as part of the second groove, in which the projection is received. The arrangement of the permanent locking recess and projection is such that the projection cannot be removed from the recess without a considerable effort on the part of the user. It cannot be removed simply by relative rotation of the first and second members, or by applying longitudinal force to the second member. This ensures the safe covering of the needle when it is desired, with the second member in a permanent locking position.

The first groove may be provided with a temporary locking recess in which the projection is received. When the projection is engaged in the temporary locking recess the needle is covered by the second member. This temporary locking position can be used when transporting filled syringes, to prevent accidents. In order to engage the temporary locking position the user moves the first and second members away from each other in the longitudinal direction. The reverse movement is applied by the user to disengage the temporary locking position. In the temporary locking position the second member may be slightly less extended from the first member than in the permanent locking position.

The or each groove means may have a further longitudinal groove with an initial locking recess. Relative rotation of the first and second members moves the projection from the further groove into the first groove. The further groove is used for assembly of the first and second members. The spring means urges the second member to cover the needle, and so that the projection is received in the initial locking recess. This locks the second member in an initial locking position, and can be used for transit of the syringe prior to use. There is no need for a separate needle sheath. Rotation of the second member by the user moves it out of the initial locking position and into the first groove, to be ready for use.

The grooves and the projections are so shaped as to allow relative rotation of the first and second members in only one direction. This adds to the safety of the device. The grooves preferably have one radial wall and one curved wall, with the projections being of complementary shape. The projections can therefore move out of a groove over the curved wall, but cannot move back, because of the engagement of the radial walls.

The spring means preferably comprises a compression spring acting between the inner end of the second member and an abutment on the first member. The spring may also provide an additional locking mechanism when the second member is in its permanent locking position. The additional locking mechanism conveniently comprises an oversize turn of the spring, adapted to be received in a radial groove in the first member when the second member is in its permanent locking position. This physically prevents re-compression of the spring, but the oversize turn does not affect sliding movement of the two members prior to this. The spring may also be arranged so that the oversize turn tends to enlarge on relative rotation of the two members.

The first member may be attached to the syringe in any suitable way, for example by a luer slip or luer lock connection to a hub at the forward end of the syringe barrel.

An embodiment of the invention is illustrated by way of example in the accompanying drawings, in which:

FIG. 1 is a longitudinal section through a syringe with the needlestick prevention device according to the invention;

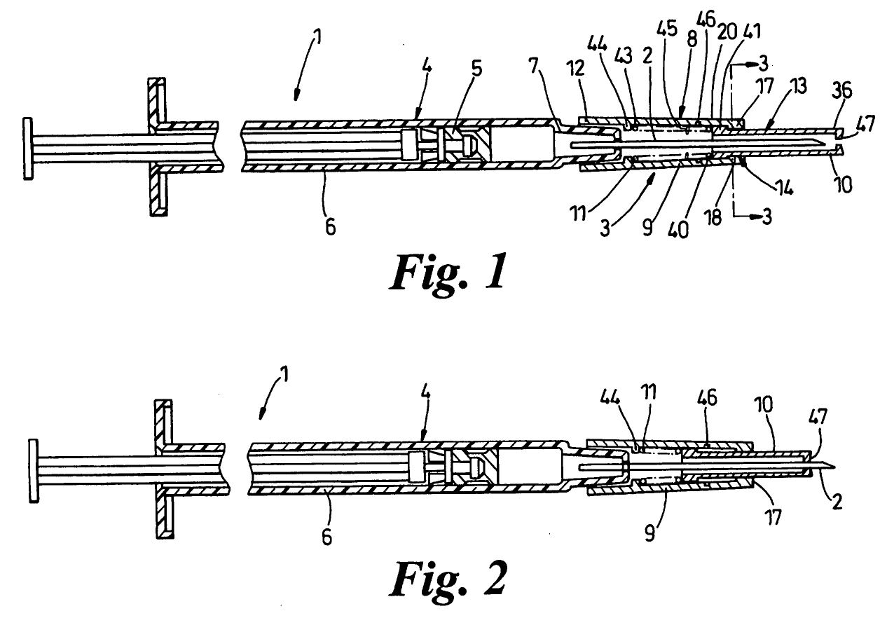

FIG. 2 is similar to FIG. 1 but shows the needlestick prevention device in a different position;

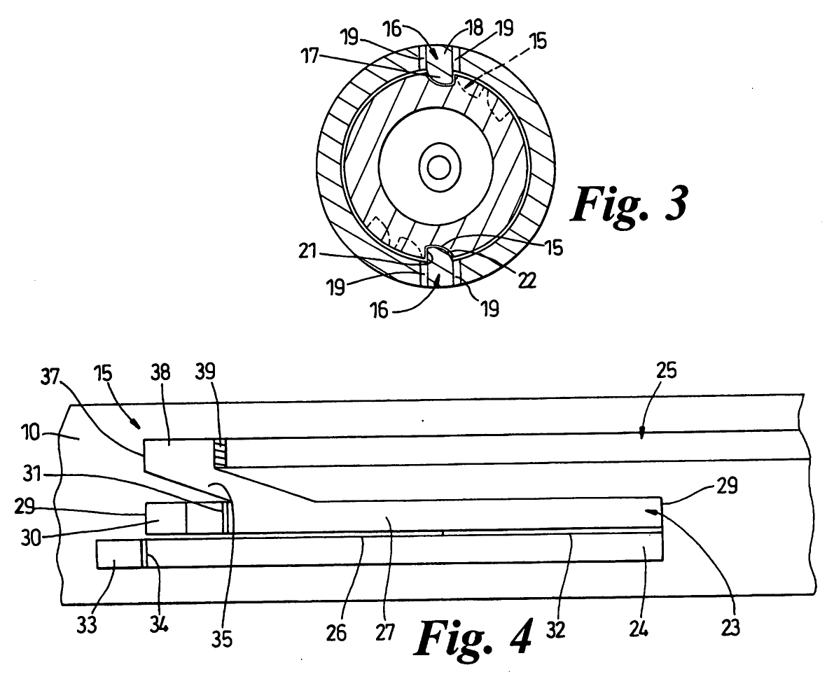

FIG. 3 is a cross-section on the line 3-3 of FIG. 1; and

FIG. 4 is a developed plan view of the exterior surface of part of the device of FIG. 1.

FIGS. 1 and 2 show a syringe 1 with a hollow needle 2 for injecting or removing fluid from a human or animal body, and a needlestick prevention device 3. The needlestick prevention device allows exposure of the needle 2 for use, and covers the needle 2 after use.

The syringe 1 is of a known type, comprising a plunger 4 with a seal 5 manually reciprocable in a cylindrical barrel 6. At its distal end the barrel 6 has a hub 7 to which the needle 2 is attached in any suitable way, while the other, proximal, end is open to allow reciprocation of the plunger 4. Movement of the plunger 4 in the barrel 6 expels or draws in fluid through the needle 2.

The needlestick prevention device 3 comprises a sheath 8 surrounding the needle 2. The sheath 8 has first and second relatively movable telescoping tubular members 9, 10 biassed apart by a spring 11. The first and second members 9, 10 are injection moulded from a plastics such as ABS or polycarbonate. The first outer member 9 is of greater diameter, and is arranged at its proximal end 12 for connection to the hub 7 of the syringe 1 in any suitable way, such as a luer slip or luer lock connection. The first outer member 9 is therefore held stationary relative to the barrel 6. The second inner member 10 is of lesser diameter, and is adapted to slide longitudinally within the first member 9, between an extended position shown in FIG. 1 in which it covers the needle 2, and a retracted position shown in FIG. 2, in which the needle 2 is exposed for use. The spring 11 biasses the second member 10 towards the extended position. The first and the second members 9, 10 are also relatively rotatable. Further, the first and second members 9, 10 have interengaging guide means 13 and locking means 14 determining movement of the second member 10.

The guide means 13 comprises groove means 15 formed in the radially outer surface of the inner member 10, and corresponding projections 16 formed on the radially inner surface of the outer member 9. As can be seen in FIG. 3, there are two diametrally opposed groove means 15 and corresponding projections 16.

Each projection 16 is formed as a radially inwardly projecting button 17 on a radially resilient arm 18. Each arm 18 is formed between two slots 19 in the distal end 20 of the first member 9. Each button 17 is shaped with a radial wall 21 and a curved wall 22, which corresponds to the shape of the groove means 15.

One groove means 15 is shown in developed form in FIG. 4, from which it can be seen that there are essentially three guide grooves 23, 24, 25 extending longitudinally of the second member from a position adjacent its proximal end 26. The grooves 23, 24, 25 each have a radial wall 27 and a curved wall 28 corresponding to the shape of the buttons 17.

The first guide groove 23 extends longitudinally for about half of the length of the second member 10, being defined by stops 29 at each end. At its proximal end the first groove 23 has a temporary locking recess 30. The button 17 can be engaged in and disengaged from the recess 30 on application of a manual force to the second member 10 in the appropriate direction, but a stop 31 prevents the engagement under the force of the spring 11. The button 17 is movable in both directions in the first guide groove 23. A wall 32 separates the first guide groove 23 from the second guide groove 24. The wall 32 has a lesser height at the distal end of the guide groove 23 to allow the button 17 to move into the second guide groove 24 on rotation of the second member 10 relative to the first member 9.

The second guide groove 24 extends parallel to the first guide groove 23. Their distal ends are aligned, but the proximal end of the second guide groove 24 is closer to the proximal end 26 of the second member 10, and has a permanent locking recess 33. The locking recess 33 is so shaped to allow the button 17 to engage in it, but to prevent its withdrawal, by means of a stop 34. The button 17 can be engaged in the recess 33 under the force of the spring 11. It will be appreciated that when the button 17 is in either the temporary locking recess 30 or the permanent locking recess 33, the second member 10 will be in an extended position covering the needle 2, but in the permanent recess 33 a greater amount of the second member 10 will project beyond the end of the needle 2.

The button 17 engages in the first guide groove 23 as a result of rotation of the second member 10 relative to the first member 9, which moves the button 17 from the third groove 25 along an inclined connecting groove 35 to an intermediate position in the first groove 23 on the distal side of the temporary locking recess 30. The third groove 25 extends from the distal end 36 of the second member 10 to a stop 37 aligned with the proximal stop 29 of the first guide groove 23. The third groove 25 allows for assembly of the two members 9, 10, and at its proximal end has an initial locking recess 38, in which the button 17 is received at the end of the assembly process, so that the second member 10 is then in the extended position. The recess 38 is formed with an abutment 39 which prevents movement of the button 17 back along the groove 25, but allows movement into the connecting groove 35 on relative rotation of then members 9, 10. The distal end 36 of the outer member 10 is formed with a curved annular flange 47 acting as a guide for the tip of the needle 2.

The spring 11 comprises a compression spring, whose distal end 40 abuts a collar 41 on the proximal end 42 of the second member 10, and whose proximal end 43 engages an abutment 44 on the proximal end 12 of the first member 9. The spring 11 also has an oversize turn 45 adjacent to its distal end 40. This turn is normally in engagement with the inner surface of the first member 9, but when the second member 10 reaches the permanent locking position with the button 17 in the recess 33, the oversize turn moves distally, and into a radial groove 46 in the first member 9 to provide an additional locking mechanism.

In use, the first and second members 9, 10 are assembled by sliding the second into the first from its proximal end 12. The spring 11 is then inserted, so that the second member 10 assumes its extended position, with the buttons 17 in the initial locking recesses 38. The second member 10 will be held in this position until it is rotated manually relative to the first member 9 through about 30°. The device 3 can then be attached to a syringe 1, and will cover the needle 2 by say 1 mm so that no further separate needle sheath is required for transit.

When the syringe 1 is to be used, the user rotates the second member 10 which brings the buttons 17 into the intermediate position in the first guide grooves 23. In this position the end of the needle 2 is uncovered, and the second member 10 can be manually retracted against the force in the spring 11 to allow for filling of the syringe 1 and expulsion of air, as well as the finding of an injection site and insertion of the needle 2. The buttons 17 are freely movable in the first guide grooves 23, so that the second member 10 can move between the retracted and intermediate positions as required by the user. If the syringe 1 is to be filled at a location remote from the patient, the user can apply a force (usually by pulling the second member 10) to engage the buttons 17 in the temporary locking recesses 30. In this temporary locking position the needle 2 is covered again by say 1 mm, so that the syringe 1 can be safely transported. To prepare the syringe 1 for injection, the second member 10 is pushed towards the first member 9 to release the temporary locking, so that the second member 10 again assumes the intermediate position with the needle 2 exposed.

The injection can then be performed, and the user twists the second member 10 again to move the buttons 17 into the second grooves 24. This preferably occurs while the needle 2 is in the patient, either before or after injection, as the user desires. On removal of the needle from the patient the force urging the second member 10 into a retracted position is released, so that the spring 11 operates to urge the second member 10 away from the first member 9, causing the buttons 17 to engage automatically in the permanent locking recesses 33. In this permanent locking position the needle 2 is covered by say 3 mm. In the permanent locking position the second member 10 cannot be moved towards the first member 9 simply by the application of a manual force. The oversize turn 45 will enter the radial groove 46, to prevent relative longitudinal movement of the members 9, 10 to provide an additional locking mechanism. The shape of the grooves and buttons allows the buttons to move as described above, but does not allow the reverse movement. In the case where a sample is being taken from a patient, the temporary locking position may be used after the sample has been taken, if it is necessary to transfer the sample from the syringe to another container using the needle. The permanent locking position will be used after transfer of the sample.

The flange 47 acts as a trap for any fluid remaining on the needle 2 after injection or the taking or transfer of a sample, if the syringe 1 is put into a position where the needle 2 is pointing down. The flange 47 may also act as a guard for the needle 2 in the permanent locking position. If the needle 2 is mounted at a slight angle, up to 5°, to the longitudinal axis of the second member 10, it will be held straight as long as it is in contact with the flange 47. The length of the flange 47 will be chosen so that, in the permanent locking position, the needle tip comes out of engagement with the flange 47. The needle 2 will then assume an angled position, and will be caught behind the flange 47, preventing it from being exposed again.

Thus, the needlestick prevention device 3 is simple to make and assemble, and provides three different locking positions in which the needle 2 is covered. The initial position allows for transit of the empty syringe, and obviates the need for a separate needle sheath. The temporary locking position allows for transport of a filled syringe (whether prior to injection or after taking of a sample), and the permanent locking position provides for safe covering of the needle 2 after final use of the syringe 1. The major advantage of the invention is that the user chooses the point at which the permanent locking occurs, and that once this choice has been made the locking happens automatically.

It will be appreciated that in a modification (not shown) the groove means could be on the first member 9 and the projections 16 on the second member 10.

Further, although the invention has been described in relation to a syringe, it can be used with other injection devices, catheters and the like.

1. A needlestick prevention device for an injection device having a hollow needle, comprising: a sheath having a first member for attachment to the injection device and a second member slidable longitudinally relative to the first member to expose or to cover the needle, and spring means biasing the second member to cover the needle, the first and second members having interengaging guide means and locking means, characterised in that the guide means include a first guide part operative to allow free longitudinal sliding movement of the second member relative to the first member, and a second guide part operative on movement by manual relative rotation of the first and second members and following release of a force urging the second member to expose the needle, in which the spring means urges the second member to cover the needle and to operate the locking means to retain the second member covering the needle.

2. A needlestick prevention device according to claim 1, in which the guide means comprises at least one groove means on one of the first and second members, and a corresponding projection on the other of the members which slides in the groove means.

3. A needlestick prevention device according to claim 2, in which two groove means and projections are provided, arranged in diametral opposition.

4. A needlestick prevention device according to claim 1, in which the second member slides inside the first member.

5. A needlestick prevention device according to claim 2, in which the or each groove means is provided on the radially exterior surface of the second member and each projections on the radially interior surface of the first member.

6. A needlestick prevention device according to claim 1, in which in the or each guide means the first guide part comprises a first groove extending longitudinally of the second member.

7. A needlestick prevention device according to claim 1, in which the second guide part comprises a second groove extending longitudinally of the second member.

8. A needlestick prevention device according to claim 7, in which the second groove is parallel to the first, and spaced from it such that a relative rotation of 30° of the members will move the projection from the first groove into the second groove.

9. A needlestick prevention device according to claim 8, in which the locking means comprises a permanent locking recess formed as part of the second groove, in which the projection is received.

10. A needlestick prevention device according claim 9, in which the first groove is provided with a temporary locking recess in which the projection is received.

11. A needlestick prevention device according to claim 10, in which in the temporary locking position the second member is slightly less extended from the first member than in the permanent locking position.

12. A needlestick prevention device according to claim 2, in which the or each groove means has a further longitudinal groove with an initial locking recess.

13. A needlestick prevention device according to claim 2, in which the grooves and projections are so shaped as to allow relative rotation of the first and second members in only one direction.

14. A needlestick prevention device according to claim 13, in which the grooves have one radial wall and one curved wall, with the projections being of complementary shape.

15. A needlestick prevention device according to claim 1, in which the spring means comprises a compression spring acting between the inner end of the second member and an abutment on the first member.

16. A needlestick prevention device according to claim 15, in which the spring also provides an additional locking mechanism when the second member is in its permanent locking position.

17. A needlestick prevention device according to claim 16, in which the additional locking mechanism comprises an oversize turn of the spring, adapted to be received in a radial groove in the first member when the second member is in its permanent locking position.

18. A needlestick prevention device according to claim 17, in which the spring is also arranged so that the oversize turn tends to enlarge on relative rotation of the two members.

19. A needlestick prevention device according to claim 1, in which the injection device is a syringe additionally comprising a barrel.

20. A needlestick prevention device according to claim 19, in which the first member is attached to the syringe by a luer slip connection to a hub at the forward end of the syringe barrel.

21. A needlestick prevention device according to claim 19, in which the first member is attached to the syringe by a luer lock connection to a hub at the forward end of the syringe barrel.
